(19) United States
(12) Patent Application Publication
(10) Pub. No.: US 2009/0202998 A1
(43) Pub. Date: Aug. 13, 2009

(54) METHOD FOR THE EXTRACTION OF BIOMOLECULES FROM FIXED TISSUES

(75) Inventors: Martin Schlumpberger, Solingen (DE); Ralf Peist, Düsseldorf (DE)

Correspondence Address: Baker Donelson Bearman, Caldwell & Berkowitz, PC, 555 Eleventh Street, NW, Sixth Floor, Washington, DC, 20004, US

(73) Assignee: Qiagen GmbH, 40724 Hilden (DE)

(21) Appl. No.: 12/097,488

(22) PCT Filed: Dec. 18, 2006
(86) PCT No.: PCT/EP2006/069842

§ 371 Date: Sep. 22, 2008

(30) Foreign Application Priority Data
Dec. 16, 2005 (DE) 10 2005 060 738.1

(51) Int. Cl. A61K 31/7088; C02F 3/34; C12Q 1/68

(52) U.S. Cl. 435/6; 435/262

(57) ABSTRACT

The present invention relates to a method for treating a fixed biological sample, comprising the steps of the method: i) provision of a fixed biological sample, ii) contacting the fixed biological sample with an aqueous solution comprising at least one nucleophilic reagent, and iii) heating the biological sample contacted with the aqueous solution. The invention also relates to the biological sample obtainable by this method, to the use of a nucleophilic reagent for the treatment of a fixed biological sample, to a kit for isolating a biomolecule from a fixed biological sample, to the use of this kit, and to a method for the treatment of a disease.

The present invention relates to a method for the treatment of a fixed biological sample, to the biological sample obtainable by this method, to the use of a nucleophilic reagent for the treatment of a fixed biological sample, to a kit for isolating a biomolecule from a fixed biological sample, to the use of this kit, and to a method for the treatment of a disease.

On removal from a living organism of biological material such as, for instance, a tissue fragment or isolated cells, the latter die after a short time unless suitable measures takes place, such as, for instance, incubation in nutrient media. The cells which have died moreover very rapidly undergo initial autolytic-fermentative and then bacterial decomposition, so that the original cell and tissue structures are destroyed. If, therefore, the intention is to remove cells or tissue fragments from an organism for histological examination, it is necessary to fix the removed biological sample in order to suppress decomposition thereof. The intention of this fixation is substantially life-like retention of living structures so that real assessment thereof is possible. The most suitable fixative must be found depending on the aim of the investigation. However, fixation also has the advantage that specimens can be stored as documents. Many morphological investigations are therefore possible only on the basis of fixed material.

The fixation is normally achieved by protein-precipitating or protein-crosslinking compounds such as acids, alcohols, ketones or other organic substances such as glutaraldehyde or formaldehyde, and fixation with formaldehyde (employed in the form of a 35% by weight aqueous solution referred to as “formalin”) followed by embedding of the fixed material in paraffin (called “formalin-fixed, paraffin-embedded” (FFPE) material) has very great importance in particular in pathology. The advantage of formalin fixation over other fixatives such as, for instance, 96% strength denatured alcohol is in particular that the cell and tissue structures are attained comparatively well in the fixation.

The disadvantage of fixation with formaldehyde is, however, in particular that as a result of the high degree of crosslinking of the biomolecules by the formaldehyde which has a crosslinking effect it is very difficult to isolate biomolecules such as, for instance, DNA, RNA or proteins from a FFPE material, but such an isolation of these biomolecules is of great importance for numerous investigations. Thus, for example, it is possible by determining the expression of the enzyme thymidylate synthase in a tumor tissue to obtain information about whether the tumor can be treated successfully with particular cytotoxic substances, or whether the tumor possibly has already developed resistances to certain cytostatics. If, for example, the mRNA which codes for the thymidylate synthase can be isolated from a FFPE tumor tissue, as described in WO-A-01/46402, a conclusion can be drawn about the enzyme expression and thus the behavior of the tumor towards certain cytostatics from the amount of mRNA in the tissue. However, since the biomolecules in the FFPE materials are in crosslinked form, and crosslinked RNA is not a suitable substrate in biochemical assays, especially in reverse transcription or the polymerase chain reaction (PCR), and crosslinked proteins often represent very poor antigens for immunological detections, it is necessary for the FFPE material to be processed appropriately for analyzing the biomolecules.

Thus, WO-A-01/46402 proposes heating the FFPE material with a chaotropic solution comprising an effective amount of a guanidinium compound at a temperature of from 75 to 100° C. for from 5 to 120 minutes. The disadvantage of this method is, however, that the conditions under which the material is heated often leads to an at least partial destruction of the biomolecules, especially sensitive biomolecules such as, for instance, RNA. Moreover the treatment times necessary for sufficient disconnection of the formaldehyde crosslinking are often very long.

US-A-2005/0014203 proposes initially heating a fixed biological sample in order to disconnect the crosslinking of the biomolecules at least partly, and subsequently to incubate the sample treated in this way with a proteolytic enzyme, for example with proteinase K, in order to decompose the tissue and the cellular structures of the tissue. The disadvantage of this method is likewise that especially with short incubation times very high temperatures are necessary to disconnect the crosslinking, and that this process takes place only very slowly at moderate temperatures.

The present invention was based on the object of overcoming the disadvantages emerging from the prior art.

The object on which the present invention was based was in particular to indicate a method with which a fixed biological sample, preferably a biological sample fixed with formaldehyde, can be treated under conditions which are as mild as possible so that it is possible in a simple manner for the biomolecules to be isolated from the biological sample or detected in the biological sample.

The present invention was also based on the object of indicating a method for treating a biological sample which is preferably fixed with formaldehyde, with which it is possible to disconnect the crosslinkings resulting in the biological sample through the fixation more quickly than in methods known from the prior art, at a treatment temperature preferably less than 95° C.

A contribution to achieving the objects mentioned at the outset is provided by a method for treating a fixed biological sample, comprising the steps of the method:
i) provision of a fixed biological sample,
ii) contacting the fixed biological sample with a preferably aqueous solution comprising at least one nucleophilic reagent, and
iii) heating the biological sample contacted with the aqueous solution.

It has surprisingly been found that the crosslinkings resulting through fixation of biological samples, especially by formaldehyde fixation, within the biological samples can be disconnected distinctly more quickly in the presence of a nucleophilic reagent.

It has further been possible surprisingly to find also that, in a particular embodiment of the method of the invention, quantitative analysis of proteins is possible from formalin-fixed tissues. The treatment differs from the known prior art in that the fixed sample is incubated at a temperature between 70 and 90° C. in the presence of a nucleophilic reagent. This leads to a release of a sufficient amount of intact proteins which can then be accurately quantified. The aqueous solution with which the fixed biological sample is contacted in the extraction of proteins preferably comprises no compound having proteolytic activity, such as, for example, a protease.

The fixed biological sample provided in step i) of the method may be a complete organism, a part of an organism, in particular a tissue fragment or a tissue section, a body fluid, a cell or a virus, with particularly preferred biological samples being cells, tissue fragments and, in particular, tissue sections. In turn, animals, plants or microorganisms such as bacteria, yeasts or fungi are suitable as organism, with a particularly preferred starting material being a human biological sample, and the most preferred biological samples being a human tissue section, a cell isolated from a human or a cultured human cell.

Fixation of the biological sample can take place with all fixatives known to the skilled worker, in particular with acids, alcohols, ketones or other organic substances such as, for instance, glutaraldehyde or formaldehyde, with formaldehyde-fixed biological samples being particularly preferred, and biological samples fixed with an aqueous formaldehyde solution comprising 1 to 35% by weight, preferably 2 to 10% by weight, formaldehyde being most preferred.

In a particularly preferred embodiment of the method of the invention, the biological sample employed in step i) of the method is a biological sample which is fixed with formaldehyde and embedded in paraffin. Such samples are normally referred to as FFPE samples. Such an FFPE sample is preferably prepared by initially dehydrating a formalin-fixed biological sample, preferably by means of an ascending alcohol series (i.e. a series of water/alcohol mixtures with increasing alcohol concentration, finally adding pure alcohol). In this connection, C₁ to C₅ alcohols are particularly preferred as alcohols, ethanol, methanol and isopropanol are further preferred, and ethanol is most preferred. The dehydrated sample is then immersed in liquid paraffin which, after the sample has been sufficiently permeated by the paraffin, is hardened. Tissue sections can then be prepared from the paraffin block by means of suitable cutting devices, for example by means of a microtome, the thickness of these sections normally being about 5 to 20 μm for examination under a light microscope. Furthermore the paraffin-embedded sample can also be reduced in size by other methods, for instance by perforating with a hollow needle or by the so-called laser capture method, to give smaller sample fragments.

In step ii) of the method, the fixed biological sample provided in step i) of the method is then contacted with a preferably aqueous solution comprising at least one nucleophilic reagent. Where the biological sample is a biological sample embedded in paraffin, it is preferred for the paraffin initially to be at least partly, preferably completely, removed from the biological sample before contacting the sample with the preferably aqueous solution. The removal of the paraffin from the biological sample can in principle take place by all methods known to the skilled worker for deparaffinization of biological samples. The deparaffinization preferably takes place by initially contacting the sample with a hydrophobic organic solvent, in particular with an aromatic hydrocarbon, most preferably with xylene, in order to dissolve out the paraffin. It may in this connection also be advantageous to agitate the mixture of the biological sample and the organic solvent, for example to shake it on a laboratory shaker, in order to ensure that the paraffin is dissolved out as efficiently as possible. The mixture is advantageously then centrifuged, and the organic solution is removed from the pellet (=biological sample). This step of dissolving out the paraffin from the biological sample can where appropriate be repeated once, twice, three times or even up to ten times. Besides dissolving out the paraffin with a suitable organic solvent, other methods are also suitable as deparaffinization methods, such as, for example, melting the paraffin, as described by Banerjee et al., Biotechniques, 18 (1995), pages 768-773.

After the removal of the paraffin it may be preferred to rehydrogenate the biological sample again, this rehydrogenation preferably taking place by stepwise washing with aqueous alcohol solutions with decreasing alcohol concentration (=descending alcohol series), with once again C₁ to C₅ alcohols being particularly preferred, ethanol, methanol and isopropanol being further preferred, and ethanol being most preferred. In a preferred embodiment of the method of the invention, an alcohol series with a concentration range from 100% by volume to 70% by volume is employed, with the concentration difference between two aqueous alcohol solutions consecutive in concentration preferably being less than 10% by volume, particularly preferably at most 5% by volume. A descending alcohol series suitable according to the invention includes for example the concentrations 100% by volume, 95% by volume, 90% by volume, 80% by volume and 70% by volume.

It is also in principle conceivable to carry out the deparaffinization and rehydrogenation with a single reagent, for example with the commercially available product EZ-DEWAX® supplied by BioGenex, California, USA.

Before the biological sample which has been deparaffinized and rehydrogenated where appropriate is now contacted in step ii) of the method with the preferably aqueous solution comprising the nucleophilic reagent, it may further be advantageous previously to dry the sample, for example by leaving to stand in the air or incubation in a drying oven.

It may further be preferred according to the invention for the biological sample which has been deparaffinized and rehydrogenated where appropriate to be homogenized before being contacted with the preferably aqueous solution in step ii) of the method, homogenization possibly being advantageous especially with larger tissue fragments. This homogenization can take place by any apparatus known to the skilled worker for reducing the size of a biological sample, in particular by high-pressure cell disruption, by means of a mechanical size-reduction apparatus, for example a mill, a rotor-stator homogenizer, an Ultra-Turrax homogenizer or a fine needle, or by ultrasonic homogenizers.

In step ii) of the method of the invention, the fixed biological sample is then contacted with a preferably aqueous solution comprising at least one nucleophilic reagent.

Suitable as nucleophilic reagent in this connection are all Lewis bases able to transfer electrons into an empty orbital or into empty orbitals of a Lewis acid. Particularly preferred Lewis bases among these are reagents which have at least one functional group which carries a negative charge, which is negatively polarized or which has at least one free electron pair.

Compound comprising a functional group having a negative charge are for example alkali metal or alkaline earth metal oxides, alkali metal or alkaline earth metal hydroxides, alkali metal or alkaline earth metal halides, alkali metal or alkaline earth metal cyanides and the like.

Reagents having at least one functional group which is negatively polarized are in particular reagents having at least one functional group in which two atoms which differ in their Alfred and Rochow electron negativity by at least 0.25, particularly preferably by at least 0.5 and further preferably by at least 1.0 are covalently connected together.

However, nucleophilic reagents which are particularly preferred according to the invention are those having at least one functional group with one or two, particularly preferably with one free electron pair, and the most preferred among these compounds in turn are those having at least one primary, secondary or tertiary amino group of the structure I

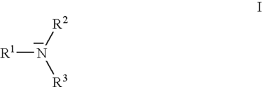

in which
R¹ is a C₁ to C₂₀ hydrocarbon group, particularly preferably a C₂ to C₁₅ hydrocarbon group and further preferably a C₂ to C₁₀ hydrocarbon group, a C₁ to C₂₀ hydrocarbon group having at least one heteroatom, a C₂ to C₁₅ hydrocarbon group having at least one heteroatom and further preferably a C₂ to C₁₀ hydrocarbon group having at least one heteroatom, or an optionally heteroatom-substituted aromatic ring system,
R² is a C₁- to C₂₀-alkyl group, particularly preferably a C₁- to C₁₀-alkyl group and further preferably a C₁- to C₂-alkyl group, in particular a methyl group or an ethyl group, a C₁- to C₂₀-hydroxyalkyl group, particularly preferably a C₁- to C₁₀-hydroxyalkyl group and further preferably a C₁- to C₂-hydroxyalkyl group, or a hydrogen atom, with a hydrogen atom being most preferred, and
R³ is a C₁- to C₂₀-alkyl group, particularly preferably a C₁- to C₁₀-alkyl group and further preferably a C₁- to C₂-alkyl group, in particular a methyl group or an ethyl group, a C₁- to C₂₀-hydroxyalkyl group, particularly preferably a C₁- to C₁₀-hydroxyalkyl group and further preferably a C₁- to C₂-hydroxyalkyl group, or a hydrogen atom, with a hydrogen atom being most preferred.

Nucleophilic reagents which are particularly preferred according to the invention and have a functional group of structure I depicted above are in particular those which have at least one functional group of structure I in which at least one of the radicals R² and R³, most preferably both radicals R² and R³ is or are a hydrogen atom. Further particularly preferred nucleophilic reagents are those having at least one functional group of structure I in which the nitrogen atom is covalently linked only to those atoms in the radicals R¹, R² and R³ which are sp³ hybridized. In particular, none of the radicals R¹, R² or R³ should be able to delocalize the free electron pair on the nitrogen atom beyond the radicals R¹, R² and R³. Thus, it is particularly preferred for none of the radicals R¹, R² and R³ to have for example structure II

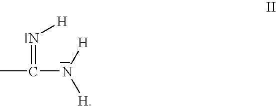

Nucleophilic reagents which are particularly preferred according to the invention and have at least one functional group of structure I are selected from the group consisting of methylamine, ethylamine, ethanolamine, n-propylamine, n-butylamine, isobutyl-amine, tert-butylamine, dimethylamine, diethylamine, diethanolamine, di-n-propylamine, diisopropylamine, dibutylamine, trimethylamine, triethylamine, triethanolamine, hexamethylenetetramine, 2-ethylhexylamine, 2-amino-1,3-propanediol, hexylamine, cyclohexylamine, 1,2-dimethoxypropanamine, 1-amino-pentane, 2-methyloxypropylamine, tri(hydroxymethyl)aminomethane, amino carboxylic acids, in particular glycine or histidine, or aminoguanidine, and among these ethanolamine, diethanolamine, triethanolamine, amino-1,3-propanediol, aminoguanidine and tri(hydroxymethyl)aminomethane are most preferred. Further preferred nucleophilic reagents having at least one functional group of structure I are aromatic amines selected from the group consisting of aniline, toluidine, naphthylamine, benzylamine, xylidene, xylene-diamines, naphthalenediamines, toluenediamines, 3,3′-dimethyl-4,4′-diphenyldiamine, phenylenediamines, 2,4′-methylenedianiline, 4,4′-methylenedianiline, sulfonyldianiline, and dimethylbenzylamine.

In a particular embodiment of the method of the invention in which the nucleophilic reagent has at least one primary amino group of structure I, the nucleophilic reagent is a C₁- to C₆-alkylamine, a C₁- to C₆-alkyldiamine, a C₁- to C₆-alkyltriamine, a C₁ to C₁₅ amino alcohol or a C₁ to C₁₅ amino diol, or a C₁ to C₁₅ amino carboxylic acid.

In another particular embodiment of the method of the invention, the nucleophilic reagent is a heterocyclic compound comprising a nitrogen atom selected from the group comprising pyrrole, pyridine, quinoline, indole, azacyclopentane, azacyclohexane, morpholine, piperidine, imidazole or a derivative of these compounds, where a derivative of these compounds preferably means a derivative in which a C₁- to C₃-alkyl group, particularly preferably a methyl group or ethyl group, is bonded instead of a hydrogen atom to one or more carbon atoms or to the nitrogen atom in the aforementioned compounds.

Particularly preferred nucleophilic reagents among those abovementioned are in particular those which are soluble in water, especially those which show a solubility of at least 1 g/L, particularly preferably at least 10 g/L and further preferably at least 100 g/L, in water at a temperature of 25° C. and at a pH of 7.

The preferably aqueous solution comprising the nucleophilic reagent described above may be based on pure, preferably deionized water or else on other aqueous systems, in particular on mixtures of water and organic solvents such as alcohols, especially mixtures of water and ethanol or methanol, with the amount of water preferably being at least 50% by weight, particularly preferably at least 75% by weight and most preferably at least 90% by weight, in each case based on the total weight of water and organic solvent, physiological saline solutions, on buffers, especially buffers comprising buffer components known to the skilled worker, such as, for example, TRIS, HEPES, PIPES, CAPS, CHES, AMP, AMPD or MOPS in an amount in a range from 0.1 to 1000 mmol/l, particularly preferably 1 to 500 mmol/l and most preferably 10 to 200 mmol/l, it being possible where appropriate for such a buffer component, depending on the structure thereof, also to serve simultaneously as nucleophilic reagent. A further possibility is also to employ nutrient media such as, for instance, MEM medium and DMEM medium, as aqueous system. The aqueous solution comprising the nucleophilic reagent is preferably prepared simply by mixing water or an appropriate aqueous system with the nucleophilic reagent.

The concentration of the nucleophilic reagent in the aqueous solution is preferably in a range from 0.1 to 10 000 mmol/l, further preferably from 1 to 5000 mmol/l, further even more preferably from 5 to 2500 mmol/l and most preferably from 20 to 1000 mmol/l. In a particularly advantageous embodiment of the method of the invention, the concentration of the nucleophilic reagent in the aqueous solution is more than 20 mmol/l, particularly preferably more than 50 mmol/l and most preferably more than 100 mmol/l.

The pH of the aqueous solution is preferably in a range from 2 to 12, particularly preferably from 4 to 9 and most preferably from 5 to 8, in each case measured at room temperature.

The contacting of the aqueous solution with the biological sample preferably takes place by immersing the biological sample in a simple manner in a sufficient amount of the aqueous solution. If the biological sample is for example in the form of a tissue section and in the form of an adherent cell on a substrate, the contacting of the biological sample with the aqueous solution preferably takes place by simply coating the substrate with the aqueous solution.

In step iii) of the method of the invention, the biological sample contacted with the aqueous solution is now heated, preferably heated to a temperature in a range from 50 to 100° C., particularly preferably from 55 to 95° C., further preferably from 60 to 90° C. and most preferably 65 to 85° C. It may be advantageous according to the invention in particular to heat the biological sample contacted with the aqueous solution to a temperature of less than 95° C., particularly preferably less than 90° C. and most preferably less than 80° C.

In a particular embodiment of the method of the invention which can be used to analyze proteins from the fixed sample, the biological sample contacted with the aqueous solution in step iii) of the method is heated to a temperature in a range from 65 to 85° C., particularly preferably from 75 to 85° C.

The biological sample is preferably boiled, i.e. heated at about 100° C., in a particular embodiment of the method of the invention which can be used for analyzing proteins from the fixed sample, for 5 to 40 min before step iii) of the method.

The duration of the heating in step iii) of the method depends essentially on the temperature and the reactivity of the nucleophilic reagent, and the skilled worker will be able to establish by simple routine tests when adequate destruction of the crosslinking of the biological sample has occurred under the given treatment conditions. However, the duration of the heating is normally in a range from 60 seconds to 10 hours, particularly preferably from 2 minutes to 5 hours and most preferably in a range from 10 minutes to 2 hours.

In a particular embodiment of the method of the invention which can be used to analyze proteins from the fixed sample, the duration of the heating can be up to 16 hours.

Depending on the nature and composition of the biological sample employed in step i) of the method, it may also be advantageous after the heating in step iii) of the method for the biological sample to be reduced in size (especially when the sample has not been reduced in size before the treatment with the nucleophilic reagent), it being possible once again to employ any device known to the skilled worker for reducing the size of biological samples. The reduction in size can take place in particular by means of high-pressure cell destruction, by means of a mechanical size-reduction device, for example rotor-stator homogenizer, a mill, an Ultra-Turrax homogenizer or a fine needle, or by ultrasonic homogenizers.

In a particular embodiment of the method of the invention, the biological sample is contacted, preferably incubated, with compounds which promotes the destruction of a biological tissue and/or the lysis of cells during the heating in step iii) of the method, before step ii) of the method or else after step iii) of the method, this compound preferably being an enzyme, a detergent, a chaotropic substance or a mixture of at least two of these components.

Enzymes preferred in this connection are in particular proteases, and among these trypsin, proteinase K, chymotrypsin, papain, pepsin, pronase and endoproteinase lys-C are particularly preferred, and proteinase K is most preferred. In a particular embodiment of the method of the invention, however, it is also possible to employ as enzyme a thermostable protease as described for instance in WO-A-91/19792 (isolated from Thermoccus celer, Thermococcus sp.AN1, Thermococcus stetteri or Thermococcus litoralis) or in WO-A-91/19792 (isolated from Staphylothermus marinus). The disclosure of these publications relating to thermostable proteases is hereby introduced as reference and forms part of the disclosure of the present invention.

In a particular embodiment of the method of the invention which can be used for analyzing proteins from the fixed sample, no compound having proteolytic activity, such as a protease, is employed. In a specific embodiment it is possible, however, to use here a nuclease such as a DNase and/or RNas, preferably a thermostable nuclease.

The concentration of the enzyme in the aqueous solution is preferably in a range from 0.001 to 5% by weight, particularly preferably 0.01 to 2.5% by weight and most preferably 0.05 to 0.2% by weight, in each case based on the total weight of the aqueous solution.

Detergents preferably employed are compounds selected from the group comprising sodiumdodecylsulfate (SDS), polyethylene glycol phenol ethers such as, for example, Triton-X-100, Tween, NP-40 or mixtures thereof, with SDS and Triton-X-100 being particularly preferred as detergents. The amount of detergent employed to lyse the cells present in the biological sample depends on the nature and amount of the biological sample and can be ascertained by the skilled worker by simple routine experiments.

It is very particularly preferred in a particular embodiment of the method of the invention which can be used to analyze proteins from the fixed sample to use SDS, sodium deoxycholate, CHAPS, Triton X100, Nonidet P40 or Tween 20 as detergent. The concentration of detergent here is preferably in a range between about 0.1-10%, particularly preferably between 1-5%.

Preferred chaotropic substances are in particular guanidinium isothiocyanate or guanidinium hydrochloride, with particular preference for guanidinium isothiocyanate. Chaotropic substances are employed in particular when nucleic acids are to be isolated from the biological sample after the treatment according to the invention. It is further preferred in this connection to employ besides the chaotropic compound also reducing compounds, in particular dithiothretil (DTT) or β-mercaptoethanol. The concentration of chaotropic compound in the treatment of the biological sample is preferably in a range from 0.1 to 50 mol/l, particularly preferably 0.5 to 20 mol/l and most preferably 1 to 10 mol/l.

Incubation of the biological material with the enzyme, the detergent or the chaotropic compound can, as explained above, take place before, during the heating in step iii) of the method, before step ii) of the method or else after step iii) of the method.

In a particular embodiment of the method of the invention, the incubation of the biological material with the enzyme, the detergent and/or the chaotropic compound takes place before step ii) of the method. For this purpose, for example, the biological sample is contacted before step ii) of the method with an aqueous solution comprising the enzyme, the detergent, the chaotropic compound or at least two thereof, where appropriate together with a reducing compound such as β-mercaptoethanol, in the form of a lysis buffer, heated to a temperature necessary for sufficient enzyme activity, and then this lysis buffer is replaced by the aqueous solution comprising the nucleophilic reagent, or else an appropriate amount of nucleophilic reagent is added to this lysis buffer.

In another particular embodiment of the method of the invention, the incubation of the biological material with the enzyme, the detergent or the chaotropic compound takes place after step iii) of the method. For this purpose, either the aqueous solution comprising the nucleophilic reagent is removed from the biological sample after the heating in step iii) of the method, and this biological sample is then contacted with the lysis buffer, or else the enzyme, the detergent, the chaotropic compound or at least two thereof, where appropriate in the form of a concentrated solution, are added to the aqueous solution comprising the nucleophilic reagent. This is followed by heating to a temperature necessary for adequate lysis. Especially when chaotropic substances are used, it is preferred for them to be added only after step iii) of the method.

In a further particular embodiment of the method of the invention, the incubation of the biological material with the enzyme, the detergent or the chaotropic compound takes place during step iii) of the method. For this purpose, the aqueous solution employed in step ii) of the method is an aqueous solution which, besides the nucleophilic reagent, comprises the enzyme, the detergent, the chaotropic compound or at least two thereof, or else the enzyme, the detergent, the chaotropic compound or at least two thereof, where appropriate in the form of a concentrated aqueous solution, is added to the aqueous solution before the heating in step iii) of the method. It is particularly preferred in this connection to employ as enzyme one of the aforementioned thermostable proteases or nucleases, because it is possible in this way to maintain the temperature for satisfactory disconnection of the crosslinkings of, preferably, 50 to 100° C., particularly preferably of 55 to 95° C., further preferably of 60 to 90° C. and most preferably 65 to 85° C. without impairing the enzyme activity.

However, irrespective of the mode of treating the tissue in steps i) to iii) of the method, ordinarily an aqueous solution in which at least part of the biomolecule dissolved out of the biological sample by the treatment according to the invention is dissolved is obtained following the heating of the biological sample. This aqueous solution may, besides the biomolecules dissolved out, also comprise further components such as, for instance, the nucleophilic reagent, enzymes, detergents, chaotropic compounds and the like.

In a particular embodiment of the method of the invention, this comprises besides steps i) to iii) of the method also the step

iv) analysis of a biomolecule dissolved out of the biological sample,
of the method, where the biomolecule is preferably a protein, a glycoprotein, a lipid, a glycolipid, an RNA or a DNA, but most preferably RNA.

In this connection, the starting composition serving for analysis of the biomolecule dissolved out of the biological sample may be
A1) the aqueous solution comprising the nucleophilic reagent which was employed in step ii) of the method and in which part of the biomolecule is present after step iii) of the method,
A2) the aqueous solution comprising an enzyme, a detergent and/or a chaotropic compound when the aqueous solution comprising the nucleophilic reagent has been replaced after step iii) of the method by an appropriate lysis buffer, or appropriate lysis components have been added to the aqueous solution comprising the nucleophilic reagent before or after step iii) of the method,
A3) an aqueous solution which has been obtained after the purification of a biomolecule in one of the two aforementioned compositions A1) or A2), for example by means of filtration, dialysis, extraction, precipitation, chromatography or a combination of these purification methods, but preferably by means of adsorption chromatography, a particularly preferred purification being in particular by separating the aqueous solution A1) or A2) into a protein fraction, an RNA fraction and a DNA fraction, for example by selective precipitation, extraction or adsorption, and separation by adsorption being most preferred, or
A4) an aqueous solution which has been obtained by extraction of the tissue obtained following step iii) of the method with an appropriate aqueous phase.

The biomolecule in the respective compositions A1) to A4) is preferably analyzed by analysis methods known to the skilled worker. Thus, suitable analysis methods are in particular immunological methods such as Western blotting, enzyme-linked immonosorbent assays (ELISAs), immunoprecipitation or affinity chromatography, mutation analyses, polyacylamide gel electrophoresis (PAGE), in particular two-dimensional polyacrylamide gel electrophoresis (2D-PAGE), high performance liquid chromatography (HPLC), polymerase chain reaction (PCR), RFLP analysis (restriction fragment length polymorphism-analysis), Southern blotting, serial analysis of gene expression (SAGE), fast protein liquid chromatography (FPLC), MALDI-TOFF spectrometry, SELDI mass spectrometry, microarray analysis and the like.

Depending on the biomolecule to be analyzed, the biological sample can be contacted with at least one enzyme during the heating in step iii) of the method, before step ii) of the method or else after step iii) of the method. The biological sample can preferably be contacted with at least one enzyme before step ii) of the method.

If the biomolecule to be analyzed is DNA and/or RNA, the enzyme may be a protease, as disclosed above. If the biomolecule to be analyzed is a protein, the enzyme may be a nuclease, as disclosed above.

A particular embodiment according to the invention comprises a method for the treatment of a biological sample fixed with formaldehyde for a later analysis of the proteins contained in the sample, comprising the steps of the method:
i) provision of a fixed biological sample,
ii) contacting the fixed biological sample with an aqueous solution comprising at least one nucleophilic reagent, and
iii) heating the biological sample contacted with the aqueous solution at a temperature in a range from 65 to 85° C.,
iv) analysis of a biomolecule dissolved out of the biological sample.

It is particularly preferred for a detergent such as SDS to be added in step ii) of the method and/or for the detergent already to be present in the aqueous solution. The biological sample is preferably boiled in the aqueous solution for 5 to 40 min before step iii) and then preferably incubated in step iii) for a period of from 20 min to 16 h.

A contribution to achieving the objects mentioned at the outset is also made by the biological sample obtainable by the method of the invention.

The use of a nucleophilic reagent as described above, in particular a nucleophilic reagent which includes at least one primary, secondary or tertiary amino group of structure I, for treating a fixed biological sample, in particular for treating a biological sample fixed with formaldehyde, also makes a contribution to achieving the objects mentioned at the outset.

A further contribution to achieving the objects mentioned at the outset is made by a kit for isolating a biomolecule from a fixed biological sample, preferably one fixed with formaldehyde, comprising

(α1) a buffer comprising a nucleophilic reagent,
(α2) a matrix for adsorbing a biomolecule, and
(α3) where appropriate an elution buffer,
(α4) where appropriate an enzyme, and
(α5) where appropriate a chaotropic substance.

Preferred as nucleophilic reagent are in particular those reagents already mentioned at the outset as preferred reagents in connection with the method of the invention.

In a preferred embodiment of the kit of the invention, it also comprises components for lysis of a cell, in particular enzymes (α4), preferably one of the proteolytic enzymes which have been mentioned above in connection with the method of the invention, detergents or chaotropic substances (α5), preferably one of the chaotropic substances which have been mentioned above in connection with the method of the invention. It is possible in particular for the enzyme and the chaotropic substance to be already present, singly or in combination, in the buffer (α1) comprising the nucleophilic reagent. However, it is also possible for the kit to include an appropriate lysis buffer (α1′) which comprises these compounds and which can be employed to disrupt a biological sample. The kit of the invention may also include a lysis concentrate (α1″) which comprises an enzyme, a detergent or a chaotropic compound in concentrated form and which can be added to the buffer (α1) at a suitable time on use of the kit.

It is possible to use as matrix (α2) for adsorbing a biomolecule all materials known to the skilled worker for adsorbing a biomolecule, in particular a protein, a DNA or an RNA, with particular preference for cellulose-based materials, especially carboxy-functional cellulose materials or diethylaminoethyl-cellulose, agarose, or mineral supports such as silica, glass, quartz, zeolites, or metal oxides, or supports coated with ion exchanger materials. Said materials may be present for example in the form of membranes or magnetic or nonmagnetic particles. This matrix is preferably present in the kit as column material in prepacked columns or as suspensions. The nature of the matrix depends crucially on the chemical structure of the biomolecules to be analyzed, the skilled worker being aware of adsorbent materials suitable for the particular purpose of use, e.g. analysis of proteins, RNA or DNA.

The elution buffers which may be present in the kit of the invention are likewise all buffers which are known to the skilled worker and which are normally employed for elution in column chromatography. The elution buffer is preferably an aqueous salt solution, in particular aqueous solutions comprising alkali metal halides such as, for instance, NaCl, KCl or LiCl, alkaline earth metal halides such as, for instance, CaCl₂ or MgCl₂, ammonium salts such as, for instance, ammonium chloride or ammonium sulfate or mixtures of at least two of these salts, it also being possible for the elution buffer where appropriate to comprise buffer systems such as, for instance, alkali metal acetate/acetic acid or buffer systems based on tris(hydroxymethyl)aminomethane. If the kit is employed for isolating RNA from a fixed tissue, and a silica membrane is employed as matrix, it is particularly preferred to employ water, especially RNase-free water, as elution buffer.

In a further particular embodiment of the kit of the invention, it may also comprise a washing buffer with which the matrix, after it has bound the biomolecule to be analyzed, is washed before elution with the elution buffer.

A contribution to achieving the object mentioned at the outset is further made by the use of the kit of the invention in a method for isolating biomolecules from a fixed biological sample, preferably one fixed with formaldehyde.

A contribution to achieving the objects mentioned at the outset is finally made also by a method for treating a disease, comprising the steps of the method:
(β1) removal of a biological sample from an organism, preferably from a mammal, particularly preferably from a human or from an animal,
(β2) fixation of the biological sample, preferably with formaldehyde,
(β3) analysis of a biomolecule from the fixed biological sample by the method of the invention including step iv) of the method,
(β4) diagnosis of a disease on the basis of the results of the analysis, and
(β5) therapeutic treatment of the diagnosed disease.

Preferred as biological sample and as biomolecule are once again those biological samples and biomolecules which have already been described at the outset in connection with the method of the invention.

The invention is now explained in more detail by means of non-limiting figures and examples.

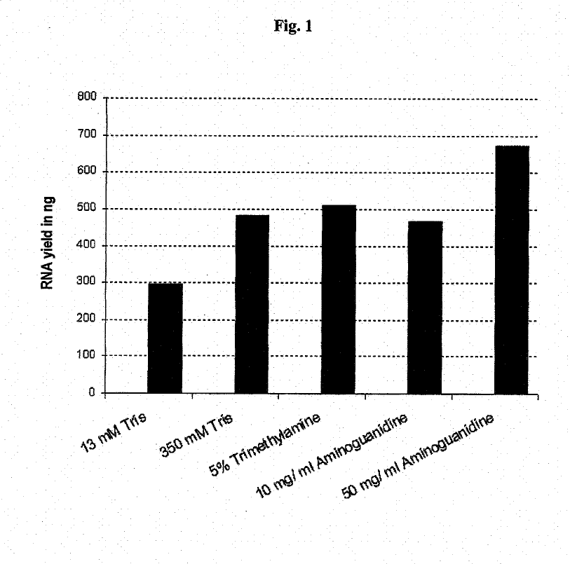

FIG. 1 shows the yield of RNA (in ng) from a formalin-fixed and deparaffinized tissue section from the lung of a rat after the treatment according to the invention.

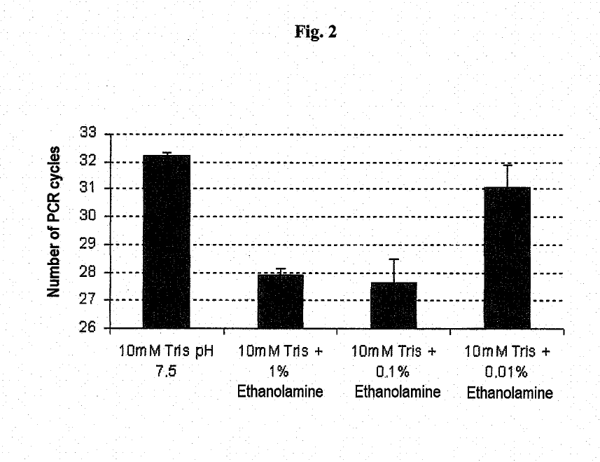

FIG. 2 shows the behavior of the RNA (in the number of PCR cycles in a real-time RT-PCR analysis which are necessary to reach a particular amount of DNA) which has been isolated by the method of the invention from a formalin-fixed and deparaffinized tissue section from the liver of a rat, in a PCR analysis.

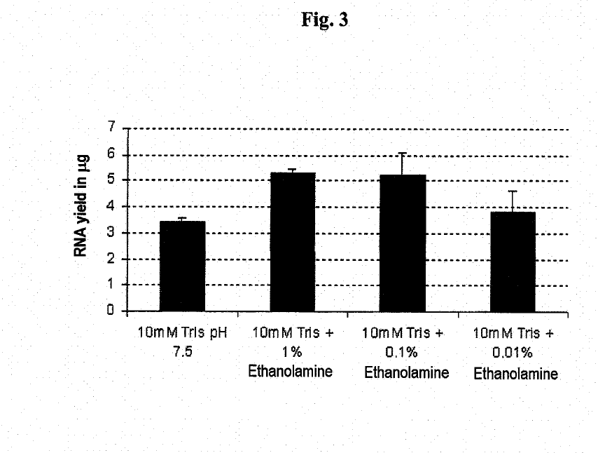

FIG. 3 shows the yield of RNA (in ng) from a formalin-fixed and deparaffinized tissue section from the liver of a rat after the treatment according to the invention.

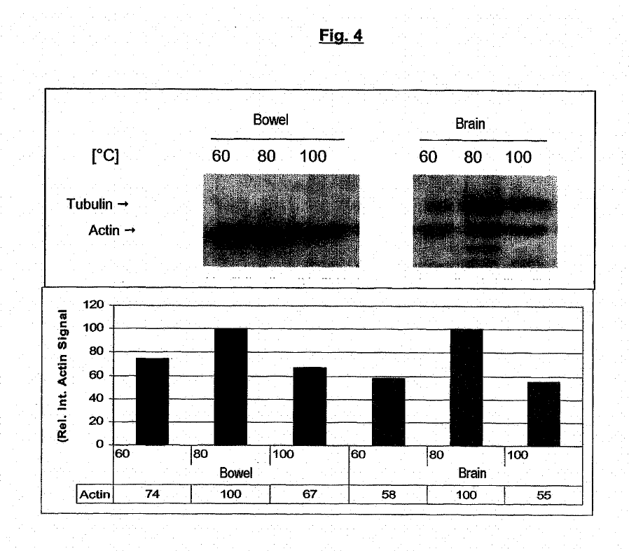

FIG. 4 shows the yield of intact protein from a formalin-fixed and deparaffinized tissue section from the bowel and the lung of a rat after the treatment according to the invention.

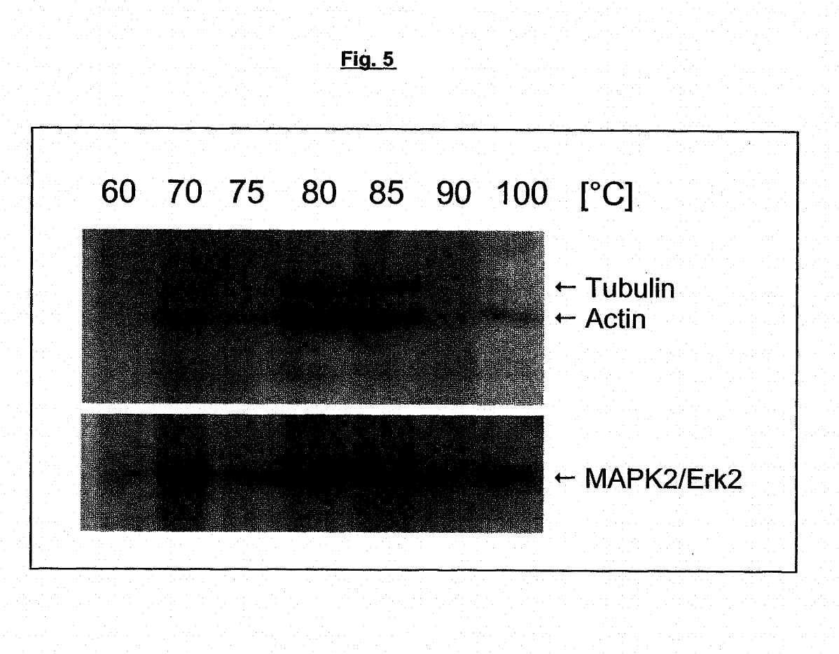

FIG. 5 shows the yield of intact proteins from a formalin-fixed and deparaffinized tissue section from the heart of a rat after the treatment according to the invention as a function of the temperature in the incubation step.

### EXAMPLES

### Example 1

Formalin-fixed and deparaffinized tissue sections (10 μm) from the lung of a rat were contacted with in each case 250 μl of an aqueous solution comprising 5 mol/l guanidine hydrochloride and in each case different nucleophilic reagents (13 mM TRIS, 350 mM TRIS, 5% by weight trimethylamine, 10 mg/ml aminoguanidine or 50 mg/ml aminoguanidine), the pH of the aqueous solutions being 7.5 in each case. The sections were incubated with the appropriate solutions at 70° C. for 60 minutes.

The RNA was isolated from the resulting cell lysate. For this purpose, initially genomic DNA was removed by means of the gDNA eliminator mini spin column (Qiagen, Hilden, Germany), the flow-through was collected and mixed with 400 μl of ethanol, and the composition obtained in this way was loaded on to an RNeasy® MinElute Spin Column (Qiagen, Hilden, Germany). After washing twice with the RPE buffer contained in the RNeasy® Kit, the membrane was dried and the RNA was eluted with 30 μl of RNase-free water. The amount of RNA in the eluate was determined by measuring the OD at 260 nm. The results are depicted in FIG. 1.

### Example 2

Formalin-fixed and deparaffinized tissue sections (10 μm) from the liver of a rat were contacted with in each case 250 μl of an aqueous solution comprising different nucleophilic reagents (10 mM TRIS, 10 mM TRIS and 1% by weight ethanolamine, 10 mM TRIS and 0.1% ethanolamine, and 10 mM TRIS and 0.01% ethanolamine), the pH of the aqueous solutions being 7.5 in each case. The cells were incubated with the appropriate solutions at 70° C. for 60 minutes. Then guanidine hydrochloride was added in a final concentration of 5 mol/l.

The RNA was isolated from the resulting cell lysate, and the behavior of the RNA in the PCR was determined by a TaqMan analysis, employing the QuantiTect probe RT-PCR kit from Qiagen, Hilden, Germany, and an appropriate primer/probe set. For this purpose, initially genomic DNA was removed by means of the gDNA eliminator mini spin column (Qiagen, Hilden, Germany), the flow-through was collected and mixed with 400 μl of ethanol, and the composition obtained in this way was loaded on to an RNeasy® MinElute Spin Column (Qiagen, Hilden, Germany). After washing twice with the RPE buffer contained in the RNeasy® Kit, the membrane was dried and the RNA was eluted with 30 μl of RNase-free water. The results of the PCR are depicted in FIG. 2. Besides the analysis of the behavior of the RNA in the PCR, also the amount of isolated RNA was determined by measuring the OD at 260 nm in example 2. The results are depicted in FIG. 3.

FIGS. 2 and 3 reveal that large amounts of RNA can be isolated by the treatment according to the invention of the samples, and the isolated RNA moreover shows good behavior in PCR analysis.

### Example 3

### Verification of the Temperature Range for Quantitative Extraction of Intact, Full-Length Proteins from Formalin-Fixed, Paraffin-Embedded Tissue

Formalin-fixed and deparaffinized tissue sections (10 μm) from bowel and brain of a rat were contacted with in each case 250 μl of an aqueous solution comprising nucleophilic reagents 1.25 M Tris/HCl (pH 6.8) and 4% SDS. The sections were boiled at 100° C. for 20 min and then incubated at 80° C. for 2 hours.

The incubation step was carried out at different temperatures for the example of two different tissues. Subsequently, equal volumes of the resulting lysates underwent Western blot analysis. The signal for actin was measured by densitometry using Easy win and was compared. The signal of the 80° C. samples were always set equal to 100% in this case.

The samples extracted at 80° C. showed the strongest signals both for actin and for tubulin in the various tissues.

### Example 4

The temperature differences of the second incubation step were chosen to be smaller in this example (brain, rat). The experiments were carried out in analogy to the protocol described in example 3.

It is clearly evident that the strongest signals are to be detected in the samples incubated in a range from 70 to 85° C., in particular at 80° or 85° C.

1. A method for treating a fixed biological sample, comprising:
i) providing a fixed biological sample,
ii) contacting the fixed biological sample with a solution comprising at least one nucleophilic reagent, and
iii) heating the biological sample contacted with the solution.

2. The method as claimed in claim 1, where the fixed biological sample is a biological sample fixed with formaldehyde.

3. The method as claimed in claim 2, where the biological sample fixed with formaldehyde is a biological sample fixed with formaldehyde and embedded in paraffin.

4. The method as claimed in claim 3, where the paraffin is at least partly removed before contact with the solution.

5. The method as claimed in claim 1, where the nucleophilic reagent is a compound which includes at least one functional group which carries a negative charge, which is negatively polarized or which includes at least one free electron pair.

6. The method as claimed in claim 1, where the nucleophilic reagent is a compound which includes at least one primary, secondary or tertiary amino group of formula I 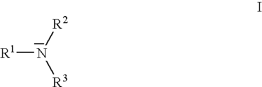
in which
R¹ is a C₁ to C₂₀ hydrocarbon group, a C₁ to C₂₀ hydrocarbon group including at least one heteroatom, or an optionally heteroatom-substituted aromatic ring system,
R² is a C₁- to C₂₀-alkyl group, a C₁- to C₂₀-hydroxyalkyl group or a hydrogen atom, and
R³ is a C₁- to C₂₀-alkyl group, a C₁- to C₂₀-hydroxyalkyl group or a hydrogen atom.

7. The method as claimed in claim 6, where at least one of the residues R² and R³ is a hydrogen atom.

8. The method as claimed in claim 6, where both R² and R³ is a hydrogen atom.

9. The method as claimed in claim 5, where the nucleophilic reagent is a C₁- to C₆-alkylamine or a C₁- to C₁₅-amino alcohol, amino diol or an amino carboxylic acid.

10. The method as claimed claim 1, where the nucleophilic reagent is a heterocyclic compound comprising a nitrogen atom or a derivative thereof, said heterocyclic compound being, selected from the group consisting of pyrrole, pyridine, piperidine, quinoline, indole, azacyclopentane, azacyclohexane, and morpholine.

11. The method as claimed in claim 1, where the nucleophilic reagent is selected from the group consisting of ethanolamine, diethanolamine, triethanolamine, amino-1,3-propanediol, aminoguanidine and tri(hydroxymethyl)aminomethane.

12. The method as claimed in claim 1, where the nucleophilic reagent has a solubility in water of at least 1 g/l at a temperature of 25° C. and at a pH of 7.

13. The method as claimed in claim 1, where the nucleophilic reagent is present in a concentration in a range from 0.1 to 10 000 mmol/l in said solution.

14. The method as claimed in claim 1, where said heating comprises heating to a temperature in a range from 50 to 100° C.

15. The method as claimed in claim 1, where said heating comprises heating for a period in a range from 60 seconds to 10 hours.

16. The method as claimed in claim 1, further comprising
iv) analyzing a biomolecule dissolved out of the biological sample.

17. The method as claimed in claim 16, where the biomolecule is RNA, DNA and/or a protein.

18. The method as claimed in claim 16, where the biological sample is contacted with at least one enzyme before, during and/or after said heating.

19. The method as claimed in claim 18, where the enzyme is a protease when the biomolecule is DNA and/or RNA.

20. The method as claimed in claim 17, where the enzyme is a nuclease when the biomolecule is a protein.

21. A biological sample obtainable by the method as claimed in claim 1.

22. A nucleophilic reagent capable of treating a fixed biological sample according to a method of claim 5.

23. A kit for isolating a biomolecule from a fixed biological sample, comprising
(α1) a buffer comprising a nucleophilic reagent,
(α2) a matrix for adsorbing a biomolecule, and
(α3) where appropriate an elution buffer,
(α4) where appropriate an enzyme, and
(α5) where appropriate a chaotropic substance.

24. The kit as claimed in claim 23, further comprising a washing buffer (α6).

25. A method for isolating biomolecules from a fixed biological sample comprising utilizing a kit according to claim 23.

26. A method for treating a disease, comprising:
(β1) removing a biological sample from an organism,
(β2) fixing the biological sample with formaldehyde,
(β3) analyzing a biomolecule from the formaldehyde-fixed biological sample by the method as claimed in claim 19,
(β4) diagnosing a disease on the basis of the results of the analysis, and
(β5) implementing therapeutic treatment of the diagnosed disease.

27. A method for treating a formaldehyde-fixed biological sample for later analysis of protein present in the sample, said method comprising:
providing a fixed biological sample,
contacting the fixed biological sample with an aqueous solution comprising at least one nucleophilic reagent,
heating the biological sample contacted with the aqueous solution at a temperature in a range from 65 to 85° C.,
analyzing a biomolecule dissolved out of the biological sample.

28. The method as claimed in claim 27, where the aqueous solution comprises a detergent.

29. The method as claimed in claim 27, where the biological sample is boiled in the aqueous solution for 5 to 40 min.

30. The method as claimed in claim 27, where the biological sample is incubated for a period of from 20 min to 16 h.

31. A method of claim 1 where the solution is aqueous.
